(19) United States
(12) Patent Application Publication
(10) Pub. No.: US 2011/0112463 A1
(43) Pub. Date: May 12, 2011

(54) COMPOSITIONS AND METHODS FOR TREATING A NEURONAL INJURY OR NEURONAL DISORDERS

(76) Inventors: JERRY SILVER, Bay Village, OH (US); Stefan Herlitze, Bochum (DE); Warren Alilain, Cleveland, OH (US)

(21) Appl. No.: 12/944,979

(22) Filed: Nov. 12, 2010

Related U.S. Application Data
(60) Provisional application No. 61/260,684, filed on Nov. 12, 2009.

(51) Int. Cl. A61M 37/00

(52) U.S. Cl. 604/20

(57) ABSTRACT

A method of improving the efficacy of denervated, quiescent, or dormant motor neurons includes expressing light sensitive G protein coupled receptors in the motor neurons, the light sensitive G protein coupled receptors modulating cellular activity in the motor neurons upon exposure to a wavelength of light and exposing the motor neurons expressing the light sensitive G protein coupled receptors to the wavelength of light.

### RELATED APPLICATION

This application claims priority from U.S. Provisional Application No. 61/260,684, filed Nov. 12, 2009, the subject matter, which is incorporated herein by reference.

### TECHNICAL FIELD

This application relates to a method of treating a neuronal injury and/or neuronal disorder, and more particularly relates to a method of treating a neuronal injury and/or neuronal disorder using a light sensitive transmembrane protein.

### BACKGROUND

G-protein coupled receptors (GPCRs) constitute a major class of proteins responsible for transducing a signal within a cell. GPCRs have three structural domains: an amino terminal extracellular domain, a seven transmembrane domain containing seven transmembrane domains, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

The GPCR protein superfamily can be divided into five families: Family I, receptors typified by rhodopsin and the β-2-adrenergic receptor and currently represented by over 200 unique members (Dohlman et al., Annu. Rev. Biochem. 60:653-688 (1991); Family II, the parathyroid hormone/calcitonin/secretin receptor family (Juppner et al., Science 254:1024-1026 (1991); Lin et al., Science 254:1022-1024 (1991); Family III, the metabotropic glutamate receptor family (Nakanishi, Science 258 597:603 (1992)); Family IV, the cAMP receptor family, important in the chemotaxis and development of D. discoideum (Klein et al., Science 241:1467-1472 (1988)); and Family V, the fungal mating pheromone receptors such as STE2 (Kurjan, Annu. Rev. Biochem. 61:1097-1129 (1992)).

There are also a small number of other proteins which present seven putative hydrophobic segments and appear to be unrelated to GPCRs; they have not been shown to couple to G-proteins. Drosophila expresses a photoreceptor-specific protein, bride of sevenless (boss), a seven-transmembrane-segment protein which has been extensively studied and does not show evidence of being a GPCR (Hart et al., Proc. Natl. Acad. Sci. USA 90:5047-5051 (1993). The gene frizzled (fz) in Drosophila is also thought to be a protein with seven transmembrane domains. Like boss, fz has not been shown to couple to G-proteins (Vinson et al., Nature 338:263-264 (1989).

G proteins represent a family of heterotrimeric proteins composed of α, β, and γ subunits, that bind guanine nucleotides. These proteins are usually linked to cell surface receptors, e.g., receptors containing seven transmembrane domains. Following ligand binding to the GPCR, a conformational change is transmitted to the G protein, which causes the α-subunit to exchange a bound GDP molecule for a GTP molecule and to dissociate from the β-γ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α subunits are known in humans. These subunits associate with a smaller pool of β and γ subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

### SUMMARY

This application relates to a method of improving the functional efficacy of a denervated, quiescent, or dormant motor neuron. The method includes expressing one or more light sensitive G protein coupled receptors in the motor neuron. The one or more light sensitive G protein coupled receptors can modulate cellular activity in the motor neuron upon exposure to a wavelength of light. The method further includes exposing the motor neuron expressing the one or more light sensitive G protein coupled receptors to the wavelength of light.

Another aspect of the application relates to a method of treating a central nervous system injury. The method includes expressing one or more light sensitive G protein coupled receptors in motor neurons that affect an impaired motor function. The one or more light sensitive G protein coupled receptors can modulate cellular activity in the motor neurons upon exposure to a wavelength of light. The method further includes exposing the motor neurons expressing the one or more light sensitive G protein coupled receptors to the wavelength of light.

A further aspect of the application relates to a method of restoring functional breathing in a subject with a CNS injury. The method includes expressing one or more light sensitive G protein coupled receptors in motor neurons that affect functional breathing in the subject. The one or more light sensitive G protein coupled receptors can modulate cellular activity in the motor neurons upon exposure to a wavelength of light. The method further includes exposing the motor neurons expressing the one or more light sensitive G protein coupled receptors to the wavelength of light.

Yet another aspect of the application relates to a method of improving bladder function in a subject. The method includes expressing one or more light sensitive G protein coupled receptors in neurons that affect the bladder function. The one or more light sensitive G protein coupled receptors can modulate cellular activity in the neurons upon exposure to a wavelength of light. The method further includes exposing the neurons expressing the one or more light sensitive G protein coupled receptors to the wavelength of light.

Yet another aspect of the application relates to a method of treating neuropathic pain in a subject. The method includes expressing one or more light sensitive G protein coupled receptors in neurons that affect the neuropathic pain. The one or more light sensitive G protein coupled receptors can modulate cellular activity in the neurons upon exposure to a wavelength of light. The method further includes exposing the neurons expressing the one or more light sensitive G protein coupled receptors to the wavelength of light.

Another aspect of the application relates to a method of promoting neuronal regeneration in a subject. The method includes expressing one or more light sensitive G protein coupled receptor in the subject's neurons affecting neuronal regeneration. The one or more light sensitive G protein coupled receptors can modulate cellular activity in the neurons upon exposure to a wavelength of light. The method further includes exposing the neurons expressing the one or more light sensitive G protein coupled receptors to the wavelength of light. The method also includes administering to the subject chondroitinase ABC in an amount effective to promote neuronal regeneration.

### BRIEF DESCRIPTION OF THE DRAWINGS

FIG. 1 illustrates the expression of ChR2-GFP in cervical spinal cord neurons after injection of a Sindbis virus into C2 hemisected animals. FIG. 1A, is a schematic of the C2 hemisection (black line), crossed phrenic pathway (dashed green lines), and ChR2-GFP photostimulation treatment protocol. After C2 hemisection, bulbospinal inputs to the ipsilateral phrenic nucleus are interrupted resulting in a quiescent phrenic nerve (red lines) and paralysis of the ipsilateral hemidiaphragm. At the same time of lesioning, ipsilateral C3-C6 spinal neurons, including contralateral projecting interneurons, are infected with a Sindbis virus to express ChR2 and GFP. After 4 d, the C3-C6 spinal cord is exposed to light to stimulate the phrenic nerve and reactivate the paralyzed ipsilateral hemidiaphragm. B, Treatment with Sindbis virus containing ChR2-GFP leads to GFP expression in ipsilateral C3-C6 spinal neurons. In addition, treatment with ChR2-GFP Sindbis virus leads to GFP expression in C3-C6 phrenic motor neurons retrogradely labeled with Dextran Texas Red. D, Dorsal; V, ventral; L, left; R, right. Scale bar, 200 μm. C, Dextran Texas Red-labeled phrenic motor neuron. Scale bar, 50 μm. D, GFP expression of Sindbis virus containing ChR2-GFP. E, Overlay of Dextran Texas Red-labeled phrenic motor neurons expressing GFP. F, Both interneurons and motor neurons infected with ChR2-GFP send neurites across or toward the midline and are in a position to potentially affect contralateral neurons and/or motor output. Arrows point to motor neuronal neurites projecting to the midline, and arrowheads point to interneuronal neurites. Scale bar, 100 p.m. G, Enlarged image (dotted line rectangle) of interneurons with midline projecting neurites.

FIG. 2 illustrates photostimulation of ChR2-GFP-expressing spinal neurons leads to a return of hemidiaphragmatic EMG activity that can be reinitiated in C2-hemisected animals and can influence the contralateral hemidiaphragm, through midline projecting spinal neurons. A, In C2-hemisected animals treated with virus containing only the GFP vector, there is no respiratory activity ipsilateral to the lesion before and after photostimulation, (only EKG activity is present). B, In C2-hemisected animals that were treated with virus containing the ChR2 and GFP vector, there is no activity before photostimulation. However, after intermittent photostimulation, there is a return of activity that is rhythmic and synchronous with the intact, contralateral side. EMG activity persisted for at least 1 min after the cessation of photostimulation. After photostimulation induced return of activity, there is a gradual cessation of EMG activity of the hemidiaphragm ipsilateral to the lesion. C, Photostimulation of spinal neurons infected to express ChR2 in C2-hemisected animals can return hemidiaphragmatic activity a number of times in the same animal, including after restored activity have ceased initially. Recovery was repeated up to five times in the same animal. D, E, in nonhemisected animals there is a significant increase of hemidiaphragmatic EMG activity contralateral to ChR2-GFP Sindbis virus injection with photostimulation (integrated EMG activity in D and raw EMG activity in E). There is a slight effect on EMG activity ipsilateral to the injection.

FIG. 3 illustrates intermittent photostimulation of ChR2-expressing spinal neurons leads to a pattern of EMG hemidiaphragmatic activity that is close to normal in C2-hemisected animals. A, before photostimulation, there is no EMG activity ipsilateral to the lesion (bottom trace). Contralateral to the lesion, there is rhythmic EMG respiratory activity (top trace). B, in the same animal, during the photostimulation protocol of 5 min off, 5 min 0.5 Hz stimulation, a trace amount of EMG activity begins to develop ipsilateral to the lesion (lower trace). As the EMG activity begins to dwindle, the contralateral, intact side begins to display an increase of EMG activity (upper trace). C, this cycling of high intensity activity that wanes, while the contralateral side increases activity, continues with each period of high intensity activity being slightly more than the last (C compared with B), and this is after the last round of photostimulation. The left two traces are of the raw EMG signal, and the right is of the same time point but integrated and rectified. Brackets under traces indicate periods between onsets of increased diaphragmatic EMG activity. D, E, Eventually EMG activity becomes closer to normal patterned respiratory EMG activity. E, inset of D. F, a trace of control-treated animal after photostimulation. G, a representative trace of the waxing and waning exhibited by non-C2-hemisected animals that expressed ChR2 and were photostimulated. Top trace is of the injected side.

FIG. 4 illustrates induction of respiratory plasticity and recovery of hemidiaphragmatic EMG activity results in increases of average peak amplitude and duration of inspiratory bursts after recovery of breathing which is NMDA receptor dependent. A, there was no change in the frequency of breaths before and after stimulation in ChR2-expressing animals, GFP-expressing animals, and MK-801-treated animals. B, after photostimulation, there was an increase of peak EMG amplitude during inspiratory bursts bilaterally in photostimulated ChR2 animals (blue bars). After blockade with MK-801, this increase was abolished (green bars) and brought back to control levels (red bars). C, after photostimulation, there was an increase in the duration of EMG inspiratory bursts bilaterally in photostimulated ChR2 animals (blue bars). After blockade with MK-801, the increase in duration was attenuated (green bars) and brought back to control levels (red bars). Measurements of postphotostimulated animals were made where normal patterned breathing had occurred, i.e., postoscillatory phasic activity. C, control, nonlesioned side; L, lesioned side.

FIG. 5 illustrates a model of light-induced activity-dependent plasticity. It is contemplated that (1) intermittent light stimulation and activation of the sodium channel ChR2 results in (2) membrane depolarization/activation followed by (3) the release of the Mg2⁺ block of the NMDA receptor, a ligand-gated Ca2⁺ channel. After release of the Mg⁺ block, (4) the resulting influx of Ca2⁺ will result in (5) induction of 2° messenger systems and cascade events, possibly insertion or phosphorylation of AMPA receptors, the primary mediator of the descending glutamatergic drive to the phrenic motor neurons, to the postsynaptic membrane or perhaps some new or unique form of activity-dependent synaptic plasticity. (6) Potentiation of the phrenic motor pool to subthreshold levels of glutamate from spared pathways/axons is achieved.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

As used herein, the terms “modulate” or “modulating” can refer to causing a change in neuronal activity, chemistry and/or metabolism. The change can refer to an increase, decrease, or even a change in a pattern of neuronal activity. The terms may refer to either excitatory or inhibitory stimulation, or a combination thereof. The terms can also be used to refer to a masking, altering, overriding, or restoring of neuronal activity.

As used herein, the term “subject” can refer to any warm-blooded mammal including, but not limited to, human beings, pigs, rats, mice, dogs, goats, sheep, horses, monkeys, apes, rabbits, cattle, etc.

As used herein, the terms “treat” or “treating” shall have their plain and ordinary meaning to one skilled in the art of pharmaceutical or medical sciences. For example, “treat” or “treating” can mean to prevent or reduce a pain in a subject.

This application relates to compositions and methods for treating nervous system injuries, neuronal disorders, neuronal injuries and to methods that can be used to modulate neuron activity and particularly neuron activity in a subject. The methods and compositions use the expression of light-sensitive (or light-activated) transmembrane proteins in neurons and methods of photostimulating such transmembrane proteins to modulate or control cellular activity.

The methods of the application provide for the ability to control via specific wavelengths of light, the activation or ion fluxes and G-protein signaling pathways in targeted neurons. It was found that the extracellular and transmembrane domains of opsins (e.g., vertebrate rhodopsin) use light energy to activate G-proteins at the intracellular site of a cell. The light-sensitive transmembrane G protein coupled receptors employed by the application include a light sensitive extracellular domain and an intracellular domain capable of modulating an intracellular signaling pathway. The intracellular regions of a GPCR determine the G protein specificity, the precise targeting of the GPCR to subcellular structures and the interaction with intracellular proteins necessary for the functional efficacy of neurons.

The expression of a light-sensitive transmembrane GPCR and subsequent photostimulation of the neurons expressing the light-sensitive GPCR can be used restore neuronal functional activity or efficacy and can be used to control neuronal activity, for example, after debilitating lesions of the CNS, which leave CNS neurons denervated and quiescent. Without being bound by theory, it is thought that neuronal activity is restored and controlled through potentiation of denervated target neurons and supersensitivity to spared axonal inputs.

One aspect of the application, therefore, relates to a method of improving the functional efficacy of neurons, such as quiescent or dormant neurons. In the method, light-sensitive transmembrane proteins are expressed from the neurons. The light sensitive transmembrane proteins modulate cell activity upon exposure to a wavelength of light. The method further includes exposing the neurons expressing the light sensitive transmembrane proteins to the wavelength of light effective to modulate activity and/or modulate cell signaling. Neurons in accordance with the application can include at least one of a motor neuron or a sensory neuron.

In an aspect of the application, the neuron expressing the light-sensitive transmembrane protein can be a motor neuron and the modulation of cell activity and/or the modulation of signaling of the motor neuron can stimulate bursting activity of the motor neuron upon exposure to light. In some aspects of the application, the modulation of cell activity can produce action potentials.

In some aspects of the application, a neuron can express a first light-sensitive G-protein coupled receptor that is activated by light having a first wavelength and once activated modulate a first cell activity. In other aspects, the neuron can express a second light-sensitive G-protein coupled receptor activated by light having a second wavelength and once activated modulating a second cellular activity. In some aspects, the second wavelength is different than the first wavelength and the second signaling pathway is different from the first signaling pathway. Activation of the respective intracellular pathways can be controlled separately or in concert depending on the wavelength(s) applied.

Examples of light-sensitive transmembrane proteins that can activate cation channels include channel rhodoposins, such as ChR1, ChR2, and ChR3 (e.g., channelrhodoposin from Chlamydomonas reinhardtii). These light-sensitive transmembrane proteins when expressed in neuronal cells, such as quiescent and dormant neuron, of a subject being treated can restore neuronal activity upon exposure to light.

By way of example, ChR2 a light activatable non-selective cation channel, which can be persistently opened during application of light, was expressed in phrenic nucleus neurons. Exposure of the transfected cell to light induced ChR2 currents in the cells, which in turn induced bursting activity in the cell.

Additional light-sensitive transmembrane proteins that can be expressed in cells to induce cellular activity or signaling include light activated ion transporters, such as bacterio rhodopsin, vertebrate and invertebrate rhodopsins, and light activated adenylate cyclase (PAC). Some aspects of the application employ light-sensitive transmembrane proteins, such as vertebrate rhodopsin 4 or halorhodopsin, that act as hyperpolarizing off-switches to inhibit or reduce cellular activity or signaling when photostimulated.

In order to control the G protein modulation of cellular activity of the application, light activated GPCRs can be used which are able to control each of the G protein coupled receptor pathways Gs, Gq and Gi/o in neuronal circuits. By choosing GPCRs, which are activated by different wavelengths of light, and mutating the intracellular regions to allow coupling to the Gi/o, Gq, and Gs pathways, activation of the corresponding pathways can be controlled. In one example, three vertebrate rhodopsin/opsin, which can be activated by UV/blue, cyan/green and yellow/red light, can be expressed on a neuron to control at least two but possibly three cell signaling pathways simultaneously. Chimeric receptors for use in the present application can be produced by standard mutagenesis techniques using PCR and Quickchange methods (STRATAGENE) as previously described in the art (Herlitze et al., (1996) Nature, 380:258-62; Herlitze and Koenen, (1990) Gene, 91:143-147; and Li et al., (2005) Proc Natl Acad Sci USA, 102:17816-21).

In an aspect of the application, the light-sensitive transmembrane proteins can be expressed in the cells using gene therapy. In an aspect of the application, the gene therapy can use a vector including a nucleotide encoding the light-sensitive transmembrane protein. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use herein include viral vectors, lipid based vectors and other non-viral vectors that are capable of delivering a nucleotide encoding a light sensitive G protein coupled receptor according to the present application to the target cells. The vector can be a targeted vector, especially a targeted vector that preferentially binds to neurons and, such as phrenic motor neurons and Onuf nucleus neurons. Viral vectors for use in the application can include those that exhibit low toxicity to a target cell and induce production of therapeutically useful quantities of the light-sensitive transmembrane protein in a cell specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the light-sensitive transmembrane protein and is replication-defective in humans.

Other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and the light-sensitive transmembrane protein nucleic acid. In methods of delivery to neoplastic cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to a light-sensitive transmembrane protein gene. These former may include the viral LTRs, a primer binding site, a polypurine tract, att sites, and an encapsidation site.

In some aspects, a lentiviral vector can be employed. Lentiviruses have proven capable of transducing different types of CNS neurons (Azzouz et al., (2002) J Neurosci. 22: 10302-12) and may be used in some embodiments because of their large cloning capacity. In one particular example, a lentiviral channelrhodopsin 2 vector controlled by the Pet-1 or FEV enhancers and a β-globulin promoter can be employed in the present methods (see Scott et al. (2005) J. Neurosci., 25:2628-36).

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety which facilitates expression of the light-sensitive transmembrane protein from the target cell.

To combine advantageous properties of two viral vector systems, hybrid viral vectors may be used to deliver a nucleic acid encoding a light-sensitive transmembrane protein to a target neuron or tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable gene expression.

Other nucleotide sequence elements, which facilitate expression of the light-sensitive transmembrane protein gene and cloning of the vector are further contemplated. For example, the presence of enhancers upstream of the promoter or terminators downstream of the coding region, for example, can facilitate expression.

In accordance with another aspect of the application, a tissue-specific promoter, can be fused to a light-sensitive transmembrane protein gene. By fusing such tissue specific promoter within the adenoviral construct, transgene expression is limited to a particular tissue. The efficacy of gene expression and degree of specificity provided by tissue specific promoters can be determined, using the recombinant adenoviral system of the present application. Neuron specific promoters such as the platelet-derived growth factor β-chain (PDGF-β) promoter and vectors are well known in the art.

In addition to viral vector-based methods, non-viral methods may also be used to introduce a nucleic acid encoding a light-sensitive transmembrane protein into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding a light-sensitive transmembrane protein into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells (e.g., neoplastic cells). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the application. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding the light-sensitive transmembrane protein can be introduced into the target cell by transfecting the target cells using electroporation techniques. Electroporation techniques are well known and can be used to facilitate transfection of cells using plasmid DNA.

Vectors that encode the expression of the light-sensitive transmembrane protein can be delivered in vivo to the target cell in the form of an injectable preparation containing pharmaceutically acceptable carrier, such as saline, as necessary. Other pharmaceutical carriers, formulations and dosages can also be used in accordance with the present application.

Where the target cell includes a motor neuron being treated, such as quiescent or dormant neurons, the vector can be delivered by direct injection at an amount sufficient for the light-sensitive transmembrane protein to be expressed to a degree, which allows for highly effective therapy. By injecting the vector directly into or about the periphery of the motor neuron, it is possible to target the vector transfection rather effectively, and to minimize loss of the recombinant vectors. This type of injection enables local transfection of a desired number of cells, especially at a site of CNS injury, thereby maximizing therapeutic efficacy of gene transfer, and minimizing the possibility of an inflammatory response to viral proteins. Other methods of administering the vector to the target cells can be used and will depend on the specific vector employed.

The light-sensitive transmembrane protein can be expressed for any suitable length of time within the target cell, including transient expression and stable, long-term expression. In one aspect of the application, the nucleic acid encoding the light-sensitive transmembrane protein will be expressed in therapeutic amounts for a defined length of time effective to induce bursting activity of the transfected cells. In another aspect of the application, the nucleic acid encoding the light-sensitive transmembrane protein will be expressed in therapeutic amounts for a defined length of time effective to restore lost function in a targeted neuron after a CNS injury.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

Certain neurons expressing a light sensitive GPCR having been exposed to light as described above may exhibit the phenomenon of bursting, in which long periods of quiescence are interrupted by a rapid firing of several spikes and a subsequent return to the quiescent state. Neuronal bursting can play important roles in communication between neurons. In particular, bursting neurons are important for motor pattern generation and synchronization. The methods of the present application have been shown to stimulate remarkable bursting activity in denervated motor neurons in a mammalian subject.

It is contemplated by the present application that chronic manipulation and stimulation of neurons or neuronal circuits through light in a subject having a central nervous system injury (e.g., a spinal cord injury) or a peripheral nervous system injury, can lead to recovery of neuronal activity and lost motor function. As shown in the Examples below, it has been demonstrated that activation of C3-C6 spinal neurons, including denervated phrenic motor neurons or interneurons, some with contralateral projections, through photo stimulation of the channel rhodopsin 2 (ChR2) protein can restore repeatedly, diaphragmatic muscle activity that is rhythmic and persistent even after the cessation of light.

Therefore, in one aspect of the application, a method of treating a central nervous system injury that results in impairment of motor function in a subject is provided. The method includes expressing light sensitive G protein coupled receptors as described above in motor neurons that affect the impaired motor function. The light sensitive G protein coupled receptors modulate cellular activity of the motor neurons upon exposure to a wavelength of light. The method further includes exposing the motor neurons expressing the light sensitive G protein coupled receptors to the wavelength of light.

Paralysis of motor function is a major consequence of spinal cord injury (SCI). Following high cervical SCI, respiratory deficits can result due to interruption of bulbospinal inputs to motor neurons innervating the diaphragm. A very small, functionally inefficient contingent of axons from the bulbospinal pathways that descend in the non-hemisected side of the cord normally re-crosses the midline caudal to the lesion to innervate the denervated, quiescent phrenic nucleus. This pathway has been termed the “crossed phrenic pathway/CPP”. It is known in the art that while physiological recovery of ipsilateral phrenic activity clearly does occur spontaneously after spinal cord injury, it has now been demonstrated that the inherent plasticity in the respiratory system that occurs without intervention, while capable of restoring some limited physiological activity in the phrenic nerve diaphragm ipsilateral to the lesion, does not result in significantly enhanced functional breathing.

As shown in the Example below, the expression of the green algae channelrhodopsin-2 (ChR2) and photostimulation in neurons can affect neuronal excitability and produce action potentials, without pre-synaptic inputs. It has been shown that in cervical spinal cord injured adult animals with spinal neurons transfected to express ChR2 followed by light stimulation results in a return of respiratory motor function. It was also shown that light stimulation of ChR2 expressing animals was sufficient to bring about recovery of diaphragmatic activity. More intense episodes of intermittent light stimulation following ChR2 expression of spinal neurons induced a dynamic type of long term respiratory plasticity that persisted long after light stimulation had ceased. In fact, intermittent photostimulation of ChR2 expressing spinal neurons was shown to lead to a pattern of EMG hemidiaphragmatic activity that is close to normal in C2 hemisected animals through a unique from of respiratory plasticity and spinal cord “learning” and adaptation.

Without being bound by theory, it is believed that the return of persistent, normally patterned breathing is due to an augmentation of the input from the normally present but latent crossed phrenic pathway. It is further believed that because cellular activity is slow to develop but, once started, builds and involves the contralateral side, the light driven activity in and around the light sensitive GPCR expressing phrenic motor pool may spread to neighboring uninfected cells. This recruitment can stimulate a large extent of the circuitry in the vicinity of activation to be more receptive to the relatively meager input from the CPP. The effect is robust and occurs in all animals although there is variability in the time it takes for the “kindling-like” episodes to begin, which in some mammals, can occur near the end of, or up to 1 hour after a 30-40 minute period of light exposure.

Therefore, the present application further relates to a method of restoring functional breathing in a subject. In the method, light-sensitive transmembrane proteins are expressed from the motor neurons in a subject that affect functional breathing in the subject. The light sensitive transmembrane proteins modulate cellular activity of the cell upon exposure to a wavelength of light. The motor neurons expressing the light sensitive transmembrane proteins are then exposed to the wavelength of light.

In some aspects, the induction of respiratory activation can be generated by patterned photoactivation locally in the phrenic motor/interneuronal pool followed by the stimulation of those that they influence within the respiratory circuit. In some aspects, the period of light exposure is intermittent. In some aspects, long length light pulses (e.g., each pulse is about 0.1 g to about 5 g for about 5 minutes to about 60 minutes) aimed at the cervical spinal cord interspersed with no-light resting periods is preferred.

It is also known that spinal cord injury, such as C2 hemisection, leads to an increase of inhibitory proteoglycans within the extracellular matrix and the perineuronal net ipsilateral to the hemisection, but distal to the cord lesion, at the level of the phrenic motor nucleus. As discussed in U.S. patent application Ser. No. 10/754,102, which is incorporated herein by reference, treatment with chondroitinase ABC (ChABC) degrades these potently inhibitory matrix molecules.

It is contemplated by the present application that enzymatically (via chondroitinase: ChABC) modifying inhibitory extracellular matrices in the PNN surrounding phrenic motor neurons combined with light induced “exercise” of the respiratory system after enzyme treatment, can maximize the sprouting capacity and functional impact of remaining nerve fibers. It is further contemplated that enhancing and/or bringing about much greater total fiber sprouting combined with enhancing the physiological output of the phrenic neurons themselves will act synergistically to improve respiration after spinal cord injury.

Therefore, in another aspect of the application, subjects can be administered chondroitinase ABC to stimulate functional respiratory plasticity in addition to the light driven GPCR (“on-switch”) method to bring about an even more enhanced amount of functional respiratory recovery than either treatment used alone. Importantly increased inhibitory matrix within the phrenic motor pool can be reduced with ChABC treatment without apparent deleterious side effects on phrenic neuron function. In some aspects of the application, bolus injections of ChABC into the vicinity of a CNS lesion can promote motor function in a subject.

The use of light-sensitive GPCRs is not only interesting for its potential to drive patterned activity and functional recovery within the denervated phrenic motor circuit but also for the additional useful side effect which may be a stimulation in the intrinsic or ChABC induced capacity for activity mediated axonal/dendritic sprouting. Therefore, in another aspect of the present application, a method of promoting neuronal regeneration in a subject is provided. The method includes expressing an effective amount of light sensitive G protein coupled receptors in the subject's targeted neurons. The light sensitive transmembrane proteins modulate cellular activity of the cell upon exposure to a wavelength of light. The motor neurons expressing the light sensitive transmembrane proteins are then exposed to the wavelength of light.

Another aspect of the application relates to a method a method of improving bladder function in a subject. Retention of urine, leading to complications such as urinary tract infection and urinary calculi, remains a major factor leading to morbidity in individuals with neurologic disorders or injury such as spinal cord injury. In high cord injury, with upper motor neuron damage, the lower nerve pathways to the bladder are intact. The aim of micturition control in these individuals is to enable them to contract the bladder musculature without direct or reflex activation of structures in the urethra (e.g., external urethral sphincter (EUS)) that may impede urine flow.

Many aspects of the storage phase of urination and some aspects of release are controlled locally within the caudal spinal cord. Bladder and EUS functions are controlled by action potentials traveling to and from spinal cord primarily, but not limited to, sacral roots, which include ventral sacral roots and dorsal sacral roots. Dorsal roots are primarily sensory (afferent nerves) to transmit sensation to spinal cord, while ventral roots primarily transmit motor pulses (efferent nerves) from spinal cord to bladder and EUS. Ventral roots and dorsal roots include both intradural nerves and extradural nerves. The intradural nerves are coupled to the spinal cord, while the extradural nerves are intertwined and are coupled to the pelvic nerves and pudendal nerve.

Therefore, it is further contemplated by the present application that a light driven substitute for one particular and critical aspect of the supraspinal control centers for micturition (e.g., those which regulate function of external urethral sphincter-EUS or bladder contraction) can be achieved using a variety of methodologies are provided in accordance with various aspects of the present application.

Accordingly, the present application also relates to the expression of light sensitive transmembrane proteins in neurons of the caudal spinal cord that affect bladder control (e.g., Onuf's nucleus neurons or) in a method of improving bladder function in a subject. The method includes expressing one or more light sensitive G protein coupled receptors in neurons that affect the bladder function. The one or more light sensitive G protein coupled receptors modulating cellular activity in the neurons upon exposure to a wavelength of light and exposing the neurons expressing the one or more light sensitive G protein coupled receptors to the wavelength of light.

In one exemplary embodiment shown in the Examples below, expressing ChR2 (on-switch) or the especially interesting vertebrate rhodopsin 4 (off switch) in neurons in and near Onuf's nucleus can improve external urethral sphincter (EUS) function after complete SCI.

In an exemplary embodiment, a viral vector including ChR2 is microinjected into the lumbosacral spinal cord of a rat. The injections are targeted to Onuf's nucleus bilaterally in the L6 to S1 spinal cord to transfect the Onuf's nucleus neurons and other nearby neurons. Following the injection, the lumbosacral spinal cords are exposed to intermittent photo stimulation leading to urination.

It is important to note that in rats, bursting activity of the EUS is a component of voiding, i.e., not a complete relaxation of the EUS. Thus, important differences exist in the lower urinary tract activation patterns between humans and rodents. In humans, the efferents function in a reciprocal way. During early urine storage, the bladder wall is quiescent and intravesical pressure remains low. However, during bladder filling, afferent reflex activity to the motor neurons gradually increases EUS contractions to maintain continence. Bladder distension at volumes sufficient to initiate micturition elicits supraspinal inhibition of EUS activity in humans (and in cats), but prolonged bursting activity of the EUS at frequencies between 6-8 Hz alternating with relaxation cycles takes place in rats. Such rhythmic contractions and relaxations of the EUS produce a pulsatile flow of urine in rodents. In higher vertebrates and humans, bursting does not occur; therefore, a complete relaxation of the EUS will be required.

Accordingly, the improved bladder function after SCI can result from either pulsatile bursting or dampening of EUS activity depending on the species of the subject. Therefore, in another aspect of the application, a GPCRs acting as an “off” switch, such as vertebrate rhodopsin 4, can be used to quiet the output from neurons in and near Onuf's nucleus and relax the EUS function in more advanced mammals, such as humans.

It is to be appreciated that both motor signals and sensory inhibition signals are but examples of signals that can be employed to evoke bladder contractions and reduce or eliminate EUS contractions. In some aspects of the application, a single signal in the form of the series of intermittent light pulses can be employed both as a motor signal for contracting the bladder and as a sensory feedback signal to subdue EUS contractions.

A variety of motor techniques can be employed to contract the bladder. For example, a variety of different continuous or intermittent light signals can be applied at the intradural nerves and/or extradural nerves of the sacral ventral root, at the pelvic nerve, at the pudendal nerve or the bladder wall to evoke bladder contraction. Alternatively, a variety of provider/subject initiated mechanical techniques can be employed to contract the bladder, for example, by distension, pressing or tapping on the skin of the human body at the location of the bladder.

In some aspects of the application, the exposure to a wavelength of light includes concurrently applying a first series of intermittent light pulses to neurons affecting external urethral sphincter (EUS) contractions and a second series of intermittent light pulses to neurons affecting bladder contractions, wherein the first and second series of intermittent light pulses are synchronized to mitigate interference with one another and to reduce or eliminate EUS contractions and evoke bladder contractions to expel urine from the subject.

In some aspects, the first and second series of intermittent light pulses have a substantially same on time for corresponding light pulses of the first and second series of intermittent light pulses. In some aspects, the first and second series of intermittent light pulses have a substantially same on time and off time period for corresponding light pulses of the first and second series of intermittent light pulses.

In accordance with the present method, a neuron can be stimulated via the GPCRs expressed on the cell by placing and/or positioning a light source in the vicinity proximate the neural cells to be stimulated. In one example, the light source can be provided in a biocompatible and/or photoconductive polymer and then locally administered to the neuron being stimulated by, for example, direct injection.

Upon positioning of the light source proximate the neural cell, the GPCRs can be activated with the appropriate wavelength of light to generate modulate the neural cell.

Exposure to a wavelength of light in accordance with the present application can be achieved by either single or multiple episodes of external light from a light source. In other aspects (e.g., in vivo methods), it is desirable to use an indwelling light source to eliminate the need for reexposure of a subjects neurons.

The light source can include a light generating means for generating light having a first wavelength effective to modulate cellular activity in a neuron via an activated light sensitive GPCR expressed on a neuron. Light from the light generating means can be used to photostimulate or photoactivate the GPCRs expressed on the cell, which then directly or indirectly stimulate or inhibit specific neuron, neural tissue, or nervous system functions. The wavelength of the light is chosen to match the photoactivation wavelength of the GPCRs. It is further contemplated that modulation of the intensity of the light source will allow the modulation of the stimulation or inhibition of the function to be controlled.

In one non-limiting example, the light source can be an in vivo fiber optic cable or LED device located in or near the targeted neuron or region of targeted neurons. Organic LEDs that give off light without heat, or thin diameter light guides that utilize water cooled LED's or fiber optic cables that have minors at the tips for deflecting light are also contemplated by the present application. The light source may also be a wireless, implantable lightsource based on electromagnetic resonant or passive radio frequency (RF) technology. A light source can also include a single monochromatic light source, such as a light-emitting diode or laser diode, or a number of such sources as described in U.S. Pat. Appl. 61/152,324, the contents of which are incorporated herein by reference.

The light source can also be biocompatible with and/or substantially non-toxic to living tissue and neural cells when positioned proximate to the cells or tissue. In some embodiments, an in vivo light source is especially advantageous since they are well tolerated and the subject does not have to be re-opened repeatedly to deliver light to the neurons expressing a light sensitive G protein coupled receptor. For example, in a method restoring functional breathing, an in vivo light source can be placed in the spinal cord just lateral to the phrenic motor pool.

In one aspect of the application, the methods of the present application can be combined with a bioluminescence system, such a luciferase system. Co-expression of luciferase and a light sensitive GPCR in accordance with the present application, such as blue-green-red light sensitive GPCRs, in a cell allow for internal activation of GPCR pathways. This is important for the treatment of living animals (e.g., humans) since the neurons can be activated by injection, intake or infusion of the luciferase ligand luciferin in a temporal manner. It will be appreciated that the bioluminescence system need not be limited to a luciferase-luciferin system and that other bioluminescence systems can be used in the application.

In one aspect, a transfected neuron can also co-express light-sensitive G-protein coupled receptor(s) and luciferase. By administering luciferin to the cell to react with the luciferase, light can be produced thereby activating the first G-protein coupled receptor. The first light-sensitive G-protein coupled receptor and the luciferase being co-expressed in neurons can be used to modulate cellular activity in the neuron.

In another aspect, a neuron of the present application can co-express a second light-sensitive G-protein coupled receptor with the first G-protein coupled receptor and the luciferase, the second light-sensitive G-protein coupled receptor can be activated by a second wavelength of light and affect a second G-protein signaling pathway.

The photostimulation of the neurons can be episodic, continuous, phasic, in clusters, intermittent, upon demand by the subject or medical personnel, or pre-programmed to respond to a sensor (e.g., a closed-loop system). The photostimulation can be operated at a constant voltage, at a constant frequency, and at a constant pulse-width. The waveform may be, for example, biphasic, square wave, sine wave, or other electrically safe and feasible combinations. Additionally, photostimulation may be applied to the neuron simultaneously or sequentially. Optimal light delivery patterns can be determined by the skilled artisan.

The ability to express of light-sensitive G protein coupled receptors to targeted cells and tissues and photostimulating the cells allows for the cell activity modulation in a number of different cell types. The light-sensitive G protein coupled receptors described above can be expressed, for example, in a heart cell via heart specific promotors for modulating the contractions (or excitability) of the heart, in the spinal cord via HB9 promotor for modulating motor neuron activity after spinal cord injury, and in neural cells or brain areas affected by degenerative diseases, such as Parkinson's disease, to control excitability in the brain area of nerve cells of choice.

Therefore, the method of the present application can be used to treat a neural injury or medical condition by neuromodulating and/or neurostimulating targeted neural cells of the subject. In the context of the present application, the term “medical condition” can refer to any movement disorders, epilepsy, cerebrovascular diseases, autoimmune diseases, sleep disorders, autonomic disorders, urinary bladder disorders, abnormal metabolic states, disorders of the muscular system, infectious and parasitic diseases neoplasms, endocrine diseases, nutritional and metabolic diseases, immunological diseases, diseases of the blood and blood-forming organs, mental disorders, diseases of the nervous system, diseases of the sense organs, diseases of the circulatory system, diseases of the respiratory system, diseases of the digestive system, diseases of the genitourinary system, diseases of the skin and subcutaneous tissue, diseases of the musculoskeletal system and connective tissue, congenital anomalies, certain conditions originating in the perinatal period, and symptoms, signs, and ill-defined conditions.

Pain treatable by the present application can be caused by conditions including, but not limited to, migraine headaches, including migraine headaches with aura, migraine headaches without aura, menstrual migraines, migraine variants, atypical migraines, complicated migraines, hemiplegic migraines, transformed migraines, and chronic daily migraines, episodic tension headaches, chronic tension headaches, analgesic rebound headaches, episodic cluster headaches, chronic cluster headaches, cluster variants, chronic paroxysmal hemicranias, hemicrania continua, post-traumatic headache, post-traumatic neck pain, post-herpetic neuralgia involving the head or face, pain from spine fracture secondary to osteoporosis, arthritis pain in the spine, headache related to cerebrovascular disease and stroke, headache due to vascular disorder, reflex sympathetic dystrophy, cervicalgia (which may be due to various causes, including, but not limited to, muscular, discogenic, or degenerative, including arthritic, posturally related, or metastatic), glossodynia, carotidynia, cricoidynia, otalgia due to middle ear lesion, gastric pain, sciatica, maxillary neuralgia, laryngeal pain, myalgia of neck muscles, trigeminal neuralgia (sometimes also termed tic douloureux), post-lumbar puncture headache, low cerebro-spinal fluid pressure headache, temporomandibular joint disorder, atypical facial pain, ciliary neuralgia, paratrigeminal neuralgia (sometimes also termed Raeder's syndrome); petrosal neuralgia, Eagle's syndrome, idiopathic intracranial hypertension, orofacial pain, myofascial pain syndrome involving the head, neck, and shoulder, chronic migraneous neuralgia, cervical headache, paratrigeminal paralysis, SPG neuralgia (sometimes also termed lower-half headache, lower facial neuralgia syndrome, Sluder's neuralgia, and Sluder's syndrome), carotidynia, vidian neuralgia, causalgia, and/or a combination of the above.

In another aspect of the present application, a method of treating neuropathic pain in a subject is provided. The method includes expressing light sensitive G protein coupled receptors in neurons that affect neuropathic pain in the subject. The light sensitive G protein coupled receptors modulate cellular activity in the neurons upon exposure to a wavelength of light. The neurons expressing the light sensitive G protein coupled receptors are then exposed to the wavelength of light.

Movement disorders treatable by the present application may be caused by conditions including, but not limited to, Parkinson's disease, cerebropalsy, dystonia, essential tremor, and hemifacial spasms.

Epilepsy treatable by the present application may be, for example, generalized or partial.

Cerebrovascular disease treatable by the present application may be caused by conditions including, but not limited to, aneurysms, strokes, and cerebral hemorrhage.

Autoimmune diseases treatable by the present application include, but are not limited to, multiple sclerosis.

Sleep disorders treatable by the present application may be caused by conditions including, but not limited to, sleep apnea and parasomnias.

Autonomic disorders treatable by the present application may be caused by conditions including, but not limited to, gastrointestinal disorders, including but not limited to gastrointestinal motility disorders, nausea, vomiting, diarrhea, chronic hiccups, gastroesophageal reflux disease, and hypersecretion of gastric acid, autonomic insufficiency; excessive epiphoresis, excessive rhinorrhea; and cardiovascular disorders including, but not limited, to cardiac dysrhythmias and arrythmias, hypertension, and carotid sinus disease.

Urinary bladder disorders treatable by the present application may be caused by conditions including, but not limited to, spinal cord injury and spastic or flaccid bladder.

Abnormal metabolic states treatable by the present application may be caused by conditions including, but not limited to, hyperthyroidism or hypothyroidism.

Disorders of the muscular system treatable by the present application can include, but are not limited to, muscular dystrophy, and spasms of the upper respiratory tract and face.

Neuropsychiatric or mental disorders treatable by the present application may be caused by conditions including, but not limited to, depression, schizophrenia, bipolar disorder, and obsessive-compulsive disorder.

As used herein, the term “headache” can refer to migraines, tension headaches, cluster headaches, trigeminal neuralgia, secondary headaches, tension-type headaches, chronic and episodic headaches, medication overuse/rebound headaches, chronic paroxysmal hemicrinia headaches, hemicranias continua headaches, post-traumatic headaches, post-herpetic headaches, vascular headaches, reflex sympathetic dystrophy-related headaches, cervicalgia headaches, caroidynia headaches, sciatica headaches, trigeminal headaches, occipital headaches, maxillary headaches, diary headaches, paratrigeminal headaches, petrosal headaches, Sluder's headache, vidian headaches, low CSF pressure headaches, TMJ headaches, causalgia headaches, myofascial headaches, all primary headaches (e.g., primary stabbing headache, primary cough headache, primary exertional headache, primary headache associated with sexual activity, hypnic headache, and new daily persistent headache), all trigeminal autonomic cephalagias (e.g., episodic paroxysmal hemicranias, SUNCT, all probable TACs, and SUNA), chronic daily headaches, occipital neuralgia, atypical facial pain, neuropathic trigeminal pain, and miscellaneous-type headaches.

### Example 1

Expression of the algal protein Channelrhodopsin-2, a rapid and light-activated cation channel, in mammalian neurons via viral gene delivery can manipulate neuronal spiking and create action potentials after light exposure in vitro. Recent studies have demonstrated that the swimming behavior of nematodes can be influenced by light activation of ionic channels and that these light sensitive channels can be expressed in living mammalian CNS tissue, where they can drive useful and functional activity within neuronal circuits.

One potential and powerful application of these dynamic light switches is in the treatment of neurological diseases and traumatic CNS injuries, in particular spinal cord injury (SCI). The disruption of descending inputs to motor neurons after SCI results in loss of motor function. It is the interruption of presynaptic inputs to motor neurons after SCI that makes it an ideal disorder model to use the ChR2 light switch and to activate these otherwise quiescent or dormant neurons because regeneration of severed axons to reinnervate target neurons and restore function is, as of now, not yet a viable therapy. In this example, we used the C2 hemisection model of SCI on adult female Sprague Dawley rats.

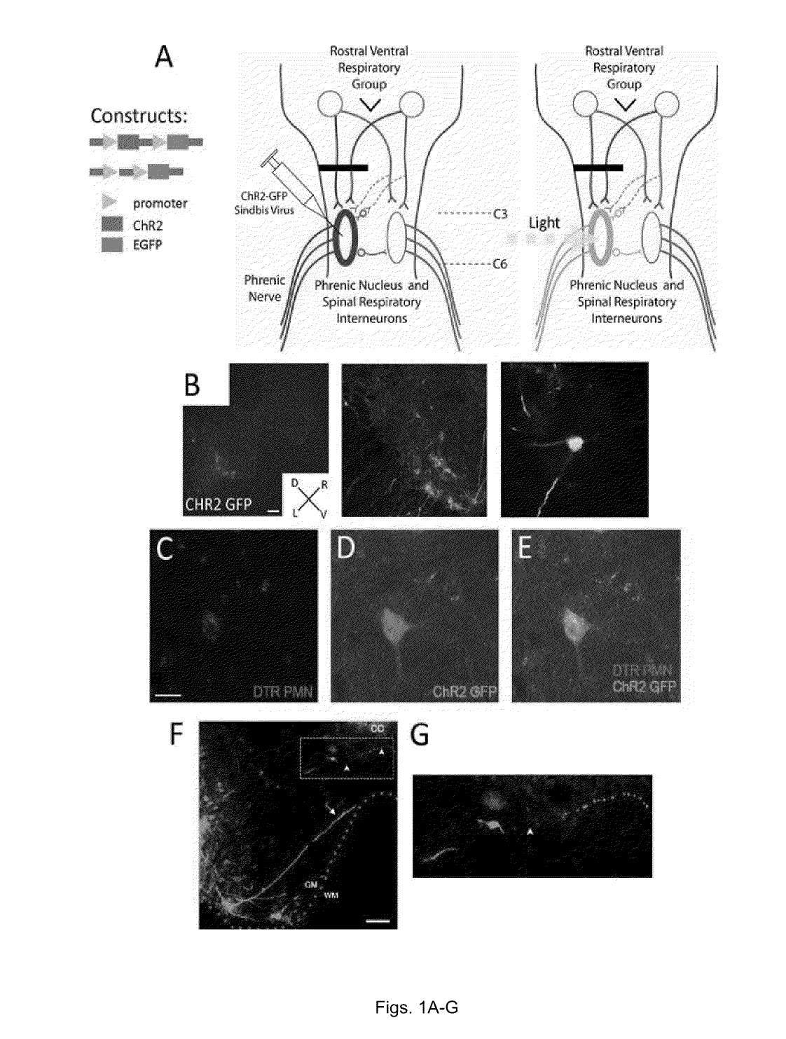

Injuries at the cervical level are one of the most common types of SCI and often result in respiratory insufficiency. In the C2 hemisection model, there is an interruption of the descending bulbospinal inputs to the ipsilateral phrenic nucleus, which innervates the hemidiaphragm, resulting in unilateral paralysis (FIG. 1A). Electromyographic (EMG) activity can be partially restored to the paralyzed hemidiaphragm through activation of an ineffective, latent pathway that arises from premotor neurons in the ventrolateral respiratory column and whose axons descend contralateral to the C2 hemisection and cross over caudal to the lesion to innervate phrenic motor neurons (PMNs) (FIG. 1A). However, spontaneous activation of this so-called “crossed phrenic pathway” is slow to develop and interventional processes to activate it can be stressful to the animal, i.e., contralateral phrenicotomy leading to asphyxiation or intermittent hypoxia.

An important advantage of ChR2 technology is that it is a relatively noninvasive technique capable of powerfully stimulating CNS circuit activity. We tested the hypothesis that after C2 hemisection and infection of spinal neurons at the level of the phrenic nucleus to express ChR2, patterned photostimulation would lead to a recovery of motor function and a return of hemidiaphragmatic activity through direct or indirect stimulation of phrenic motor neurons or potentiation of the phrenic nucleus to spared inputs.

### Materials and Methods

### C2 Hemisection and Virus Injection

Adult female Sprague Dawley rats (250-300 g) were anesthetized with a ketamine (70 mg/kg) and xylazine (7 mg/kg) solution administered intraperitoneally. After administration of the anesthetic mixture, the animals were prepared for surgery by shaving and cleansing the dorsal neck area with betadine and 70% alcohol. After the surgical prep, about a 4 cm midline incision was made on the neck. After retraction of the paravertebral muscles, a multilevel laminectomy was performed and the dura and arachnoid mater were cut with microscissors to expose several cervical segments of the animal's spinal cord. A left C2 hemisection just caudal to the C2 dorsal root was made with a sharp microblade. The hemisection was made from the midline to the lateral most extent of the spinal cord.

At the same time as hemisection, the animals received three injections of Sindbis virus (250 nl per injection) containing either the dual ChR2-GFP vector (n=14) or the green fluorescent protein (GFP) vector alone (n=9) into the C3-C6 region of the spinal cord, the level of the phrenic motor nucleus. Injections were made ipsilateral to the lesion, 0.11 cm from the midline and 0.16 cm ventral from the dorsal surface of the spinal cord, in close proximity to the phrenic nucleus, through use of a Kopf stereotaxic device. Sham/nonlesioned animals received all procedures but the hemisection (n=10). Of these 10, six received the ChR2-GFP construct, and four received control injection. Following these procedures, the paravertebral muscles were sutured back together with 3-0 vicryl and the skin stapled together with wound clips. Animals received marcaine and buprenorphine for analgesia. Saline was administered subcutaneously if the animals appeared dehydrated. The animals were housed in normal day/night schedule and given food and water ad libitum.

### Constructs and Virus

Sindbis virus vector SinRep (nsP2S⁷²⁶) and helper DH-BB were kindly provided by P. Osten (Northwestern University, Evanston, Ill.) (Kim et al., 2004). SinRep(nsP2S⁷²⁶)dSP-EGFP was constructed by subcloning another subgenomic promoter with EGFP into the ApaI site of the original SinRep(nsP2S⁷²⁶). cDNA of ChR2 (GenBank accession no. AF461397) was PCR-amplified and cloned into the XbaI and MluI sites of SinRep(nsP2S⁷²⁶)dSP-EGFP under a CMV promoter. Sindbis pseudovirions were prepared according to Invitrogen's directions (Sindbis Expression System) and then concentrated with an ultracentrifuge. Viral titer was 0.5-1×10⁸ units per ml.

### EMG Recordings and Light Stimulation

Four days after C2 hemisection and/or virus injection, the animals were anesthetized as above and prepared for light treatment and physiological recordings. In a room where all light was eliminated except for that needed to accomplish the surgical procedures, approximately an eight cm incision was made at the base of the ribcage to expose the abdominal surface of the diaphragm. Bipolar electrodes, connected to an amplifier and data acquisition set-up (CED 1401/Spike2 Data Analysis Computer Interface, Cambridge Electronic Design), were inserted into both left and right hemidiaphragms to record diaphragmatic activity. After this, the cervical area of the spinal cord was reopened again for exposure to photostimulation at a wavelength of 475 nm, i.e., blue light. The light source was a portable unit capable of producing light at various wavelengths through a fiber optic cable (Model Lambda DG-4, Sutter Instrument). Diaphragmatic motor activity was recorded before, during, and after stimulation. During recording, the animals were placed on a circulating warm water blanket to maintain body temperature. The initial protocol used for photostimulation included sustained exposure (1 min) of the C3-C6 spinal cord from the light source, as well as, intermittent exposure to light at about once per second for 1 min. In animals that received the longer lasting light delivery protocol, which resulted in more robust recovery (>1 h), the following light stimulation protocol was used: alternating 5 min rest/5 min, intermittent light stimulation for three or four cycles (30-40 min total). This protocol was adapted from the long-term facilitation induction protocol of 5 min normoxia followed by 5 min hypoxia (Fuller et al., 2003; Golder and Mitchell, 2005). The intermittent stimulation consisted of light exposure at 0.5 Hz, with each flash of light 1 s long.

NMDA Receptor Blockade with MK-801

To block NMDA receptors, 500 μl of 10 μM MK-801 (Sigma), a noncompetitive NMDA receptor antagonist, diluted in PBS, was applied to the exposed spinal cord. MK-801 was administered after 5 min of baseline recording. Recording with MK-801 continued for 5 more minutes before intermittent photostimulation and thereafter as described above.

### Data and Statistical Analysis

After recording, the raw diaphragmatic EMG signal was rectified and integrated using Spike2 software. Frequency was determined by counting total breaths for 5 min before and after photostimulation. Peak amplitude and burst duration of inspiratory bursts were measured through Spike2, for at least 25 breaths before and after stimulation. Poststimulation analyses of ChR2 animals were made during regular patterned respiratory related diaphragmatic EMG activity. All values were standardized to prestimulation measures. Statistical analysis was performed using ANOVA and Tukey's post hoc analysis. All values with a p value <0.05 were considered significant. All error bars indicate SEs.

### Fluorescence and Immunocytochemistry Analysis

For immunocytochemical experiments, phrenic motor neurons were retrogradely labeled with Dextran Texas Red (Invitrogen) at the time of hemisection. Animals were anesthetized as above and about an 8 cm incision was made at the base of the ribcage to expose the abdominal surface of the diaphragm. Five 10 μl aliquots of 0.4% Dextran Texas Red were injected into the left hemidiaphragm. The abdominal muscles were sutured together and the skin stapled together with wound clips.

Four days after injection of tracer or immediately after recording, animals were perfused first with 50 ml of PBS, followed by 250 ml of chilled 4% paraformaldehyde in PBS. The cervical spinal cord was harvested and postfixed in perfusate until sectioning. Before sectioning, a pinhole was made on the right side of the spinal cord to mark laterality. The spinal cords were sectioned transversely at 50 μm thickness on a vibratome and placed free floating in PBS.

Sections were washed three times with PBS followed by blocking in 5% NGS/0.1% BSA/0.1% Triton X-100 in PBS for 2 h at room temperature. After blocking, the sections were incubated in rabbit anti-GFP primary antibody (Invitrogen) overnight at 4° C. The next day, the sections were washed three times with PBS for 30 min each followed by incubation for 2 h at room temperature in secondary goat anti-rabbit secondary antibody conjugated to Alexa Fluor 488 (Invitrogen). After washing for three times in PBS at 30 min each, the sections were mounted with 1:1 Citifluor and PBS mounting media on slides and coverslipped. Sections were viewed and imaged on a Zeiss confocal microscope. Cell counting was accomplished by viewing every sixth section (C3-C6) with a Leica fluorescent microscope (40×). All cells containing GFP were counted for every sixth section and their numbers were totaled. The estimated cell totals per animal were derived by multiplying the value obtained above by six and then averaging the number per animal for five animals.

### Results

Adult female rats received a left C2 hemisection by incising from the midline of the spinal cord to the lateral most extent of the spinal cord, just caudal to the C2 roots. At the same time of hemisection, spinal neurons from C3-C6, the level of the phrenic nucleus, were infected with a Sindbis virus containing ChR2 (1-315) fused to GFP (FIG. 1A). The virus was injected directly into the ventral gray matter of the spinal cord (3 injections, 250 nl each, 750 nl total).

Four days after lesion and virus introduction, the C3-C6 spinal cord was exposed again and stimulated with light for physiological characterization and analysis. Before, during and after light stimulation, bilateral diaphragmatic EMG activity was recorded. Successful incorporation of the virus and ChR2-GFP protein expression in spinal neurons were verified and neuroanatomical localization of infected cells was accomplished through GFP reporter detection after physiological recordings.

### Expression of ChR2 in Adult Spinal Neurons

ChR2-GFP infection and expression was successful in the spinal cord of adult rats. GFP was expressed primarily in ventrally located spinal interneurons and motor neurons (FIG. 1B). The label was present within the cell soma but also within both the axonal and dendritic compartments. We estimated that there were ˜656±63 spinal cells infected to express ChR2-GFP per animal. A few astrocytes were also labeled with GFP. Furthermore, after retrograde labeling of PMNs by injecting Dextran Texas Red (0.4%, five times, 50 μl each injection) into the diaphragm muscle, we found that ChR2-GFP was, indeed, expressed in these particular respiratory motor neurons (FIG. 1C-E). Some motor neurons and interneurons expressing ChR2-GFP had processes that projected toward the midline (FIG. 1F). In some cases, neuritis of labeled interneurons crossed past the central canal and into the contralateral ventral horn (FIG. 1G).

### Light-Induced Stimulation of Diaphragmatic EMG Activity

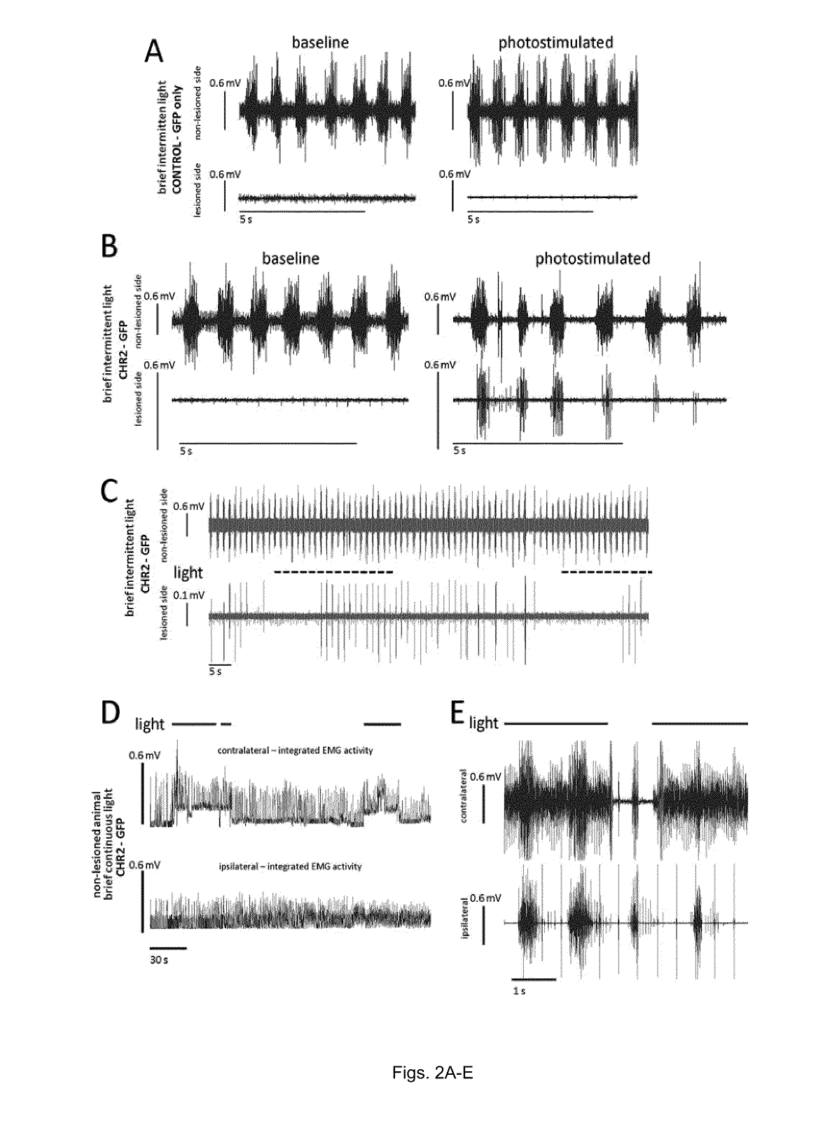

Physiological characterization of rats expressing ChR2-GFP showed that muscular activity after a cervical cord hemisection lesion could be induced in the initially paralyzed hemidiaphragm. Consistent with previous studies, there was no respiratory related EMG activity present in the hemidiaphragm ipsilateral to the lesion acutely after C2 hemisection and before photostimulation (only EKG activity was present) (FIG. 2A, B). In our first series of experiments, brief episodic or continuous periods of light stimulation were used (1 Hz, with each flash of light being 0.5 s long, total stimulation length was 30-60 s or for continuous stimulation we also used 30-60 s long exposures). Approximately 15 s after intermittent light stimulation, EMG activity was induced (FIG. 2B). The recorded activity began rhythmically and remained synchronous with respiratory hemidiaphragmatic activity contralateral to the lesion (FIG. 2B). More remarkably, the activity persisted after photostimulation had ceased and continued for up to 1 min before dwindling in magnitude and slowly ending (FIG. 2B, C). This activity was capable of being reproduced multiple times in the same animal after termination of the initial or previous instances of hemidiaphragmatic motor recovery (FIG. 2C). We attempted as many as five repetitions in the same animal, and all were successful. In control animals that received only the GFP construct, activity was absent ipsilateral to the lesion and construct injection before, during, and after photostimulation (FIG. 2A).

In contrast to the results with intermittent photostimulation, continuous episodes of stimulation (30-60 s) produced hemidiaphragmatic EMG activity in ChR2-GFP infected rats that was tonic, in that the activity was arrhythmic and nonsynchronous with the contralateral, unlesioned side. Furthermore, after termination of the continuous period of stimulation, no kind of diaphragmatic activity, rhythmic or sustained, was detectable on the side ipsilateral to the lesion (data not shown).

In control animals that were infected with ChR2-GFP and received light stimulation but did not receive a hemisection there was also considerable impact on the output of hemidiaphragmatic muscle activity (FIG. 2D, E). Bilateral EMG recordings of the diaphragm showed that during photostimulation, there was a significant increase in tonic EMG activity contralateral to the site of ChR2-GFP virus injection and infection. Interestingly, in unlesioned animals there were less significant increases of hemidiaphragmatic EMG activity ipsilateral to the expression of ChR2 (FIG. 2D, E).

### Spinal Plasticity and Adaptation in the Spinal Cord Leading to Long-Lasting Restoration of Diaphragmatic EMG Activity

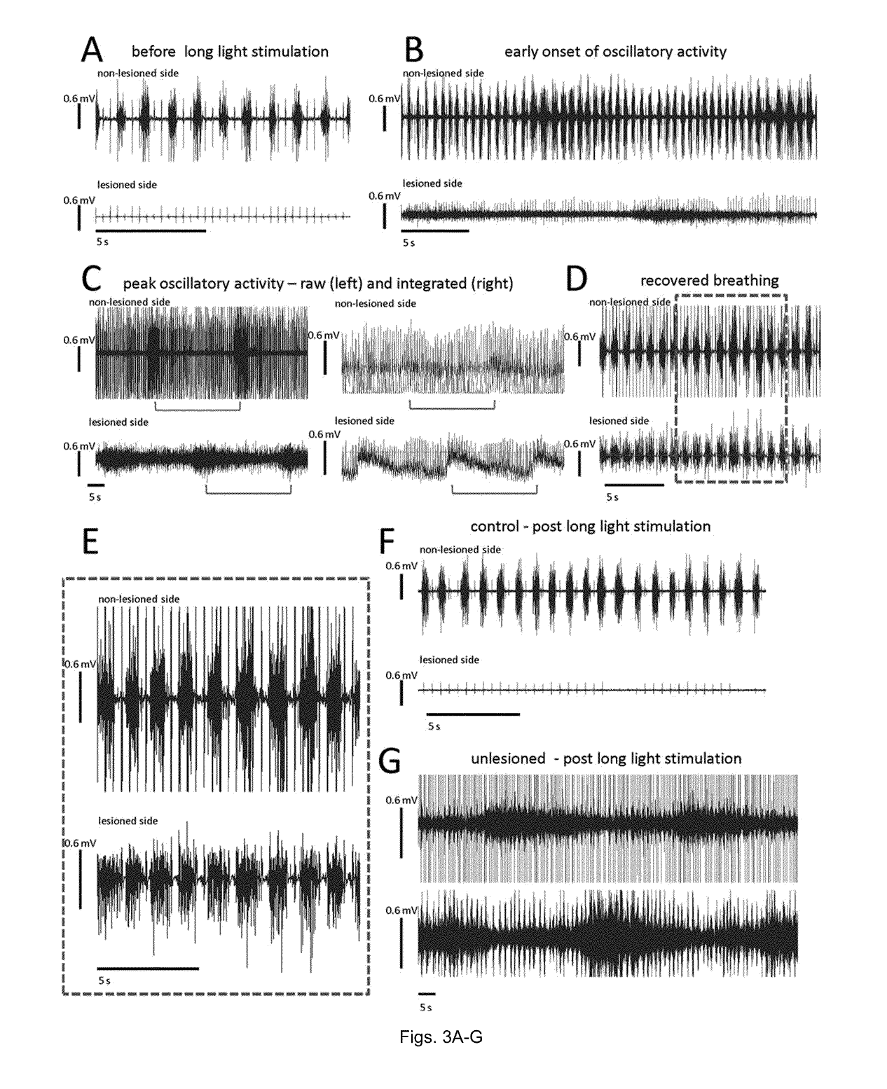

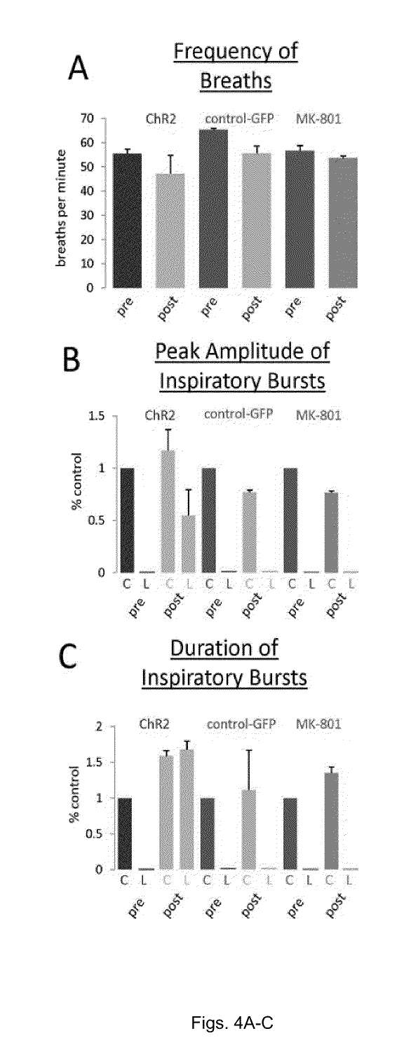

While further investigating the impact of more intense episodes of intermittent light stimulation after ChR2 expression of spinal neurons, we discovered an unusual, dynamic type of long-term respiratory plasticity that was evident in both C2 hemisected and unlesioned animals. Compared with the brief, less intense, intermittent stimulation that produced shorter lasting and relatively weak recovery in the first set of experiments; long and patterned intermittent stimulation induced long-lasting recovery. Before photostimulation, no activity was present in the hemidiaphragm ipsilateral to the lesion. However, after and sometimes even during a stimulation protocol that consisted of 5 min of baseline activity (no light), followed by 5 min of 0.5 Hz intermittent light (one second light flash, one second off) for at least three cycles, a trace amount of EMG activity would inevitably appear within the ipsilateral hemidiaphragm. This occurred between 30 and 90 min from the start of the recording session and as late as 1 h past the last round of photostimulation. The EMG bursting patterns waxed and waned in intensity repetitively in a highly regular pattern, while gradually and dramatically increasing in overall intensity compared with previous periods (FIG. 3A-C). In addition, bilateral diaphragmatic EMG recordings during these episodes showed an interesting interaction within the phrenic circuitry that controls the two sides of the diaphragm (FIG. 3B, C). As intense activity on the lesioned side would decrease, EMG activity on the opposite side would increase (FIG. 3B, C). The nonsynchronized increases in activity would oscillate until the phase onsets between the two sides coincided in 30-60 min after the last intermittent light stimulation cycle. The waxing and waning ultimately and slowly disappeared as EMG activity within the once paralyzed hemidiaphragm evolved toward a pattern that closely resembled the nonhemisected side (FIG. 3D, E). This normally patterned breathing lasted for at least 2 h in the same recording session. After ending the session and waiting 24 h before beginning another recording session, recovered breathing still persisted but at a lower magnitude. Photostimulated control animals not expressing ChR2 did not exhibit this unique pattern of respiratory output (FIG. 3F). Our analysis showed that although there was no change in frequency of breaths after light stimulation, there were significant increases in peak amplitude and burst duration during inspiratory bursts of the diaphragm bilaterally after photostimulation (FIG. 4A-C). This interesting form of respiratory plasticity was also evident in non-C2 hemisected animals (FIG. 3G). After infection and intermittent light stimulation using the 5 min protocol, oscillating waxing and waning of increasing EMG activity occurred between the two sides of the diaphragm.

### NMDA Receptor Dependence of Spinal Learning and Recovery

After application of the noncompetitive NMDA receptor antagonist MK-801 (500 μl of 10 μM MK-801 in PBS) to the exposed C3-C6 spinal cord, intermittent photostimulation failed to elicit any kind of change in diaphragmatic EMG activity both ipsilateral as well as contralateral to the ChR2 injection sites in four of six animals (FIG. 4B, C). In two animals, changes in activity did occur minimally but only contralateral to the lesioned side; and primarily it was an increase of the burst duration of every breath (FIG. 4C). The abolishment of light activated activity by MK-801 was seen in both hemisected and nonlesioned ChR2 animals.

Together, our results suggest that patterned, intermittent photostimulation can potentiate denervated phrenic motor neurons to the usually subthreshold influence of spared pathways, likely the “crossed phrenic pathway,” that remains after C2 hemisection. Potentiation of PMNs to the crossed phrenic pathway can account for the activity that persisted after cessation of light activation of ChR2, and the rhythmic breathing activity that was observed, because rhythm generation of the respiratory system is primarily supraspinal, although spinal circuits have been identified.

### Both Adult Spinal Motorneurons and Interneurons can Express ChR2 and can Influence the Contralateral Side

Our data also showed that there were changes in diaphragmatic EMG activity contralateral to the site of ChR2 expression. Interestingly, there appears to be a subset of neurons, possibly ChR2-expressing interneurons or, less likely, motor neurons that can influence contralateral phrenic motor neurons after activation with photostimulation. After further examination of GFP expression in C4 spinal cord cross sections, both interneurons and motor neurons were capable of projecting neurites toward the midline. In fact, some interneuronal processes crossed the midline within the ventral white commissure to the contralateral side. Recent anatomical studies have suggested that interneurons may play a significant role in mediating crossed phrenic activity. Our physiological data provide strong support for the functional influence of contralaterally projecting cells on phrenic motor circuitry.

These sets of experiments further suggest a sophisticated level of connectivity and circuitry related to respiration between the two sides of the spinal cord that has not been observed before in the rat. In addition to the functional bilaterality of interconnections at the level of the phrenic motor pool, the oscillating patterns of EMG activity that slowly build toward normal levels and synchrony resulting in recovery of normal patterned breathing, suggests the idea of synaptic strengthening or plasticity within spinal respiratory circuitry which can adapt and learn so that functional activity that is normal in pattern can emerge. It is also possible that our long light stimulation protocol has revealed a dormant, spinal respiratory circuit that is similar to a central pattern generator (CPG) whose activation leads to the alternating firing rhythm that develops between the two sides of the diaphragm.

Our observation that there is an increase in background or tonic activity during light stimulation suggests that a variety of spinal interneurons or possibly even glia expressing ChR2 may have inputs to the primary spinal circuitry mediating respiration. Using more specific neuronal promoters, the precise role of each cell type in the restoration of respiratory activity can be dissected. Furthermore, because there is a delay and slow augmentation in respiratory related EMG activity in animals with both brief and long light stimulation, it is possible that more widespread alterations in circuit activation via recruitment of respiratory associated spinal neurons not expressing ChR2 is required for the recovery process. It is conceivable that the episodes that initially emerge, especially after long light exposure, parallel the kinds of events that occur during the phenomenon of kindling, in which patterned, low-intensity electrical stimulation can spread to nearby circuits, leading to progressive amounts of CNS activity even after stimulation has ceased. Interestingly, kindling, which, in turn, can lead to the induction of epileptiform activity, is also partially glutamate and NMDA receptor activation associated. However, in the lesioned spinal cord, where there is a dearth of activity, light-induced “kindling” and the onset of seizure-like activity somehow become regulated in a beneficial way. This is probably because of the continuing influence of the normal respiratory rhythm being generated from the brainstem as well as the presence of relatively intact seizure dampening mechanisms within the spinal cord, including the effects of astrocytes and inhibitory interneurons.

A Model of ChR2 Activation that can Lead to Long-Lasting Recovery of Muscle Activity after Spinal Cord Injury

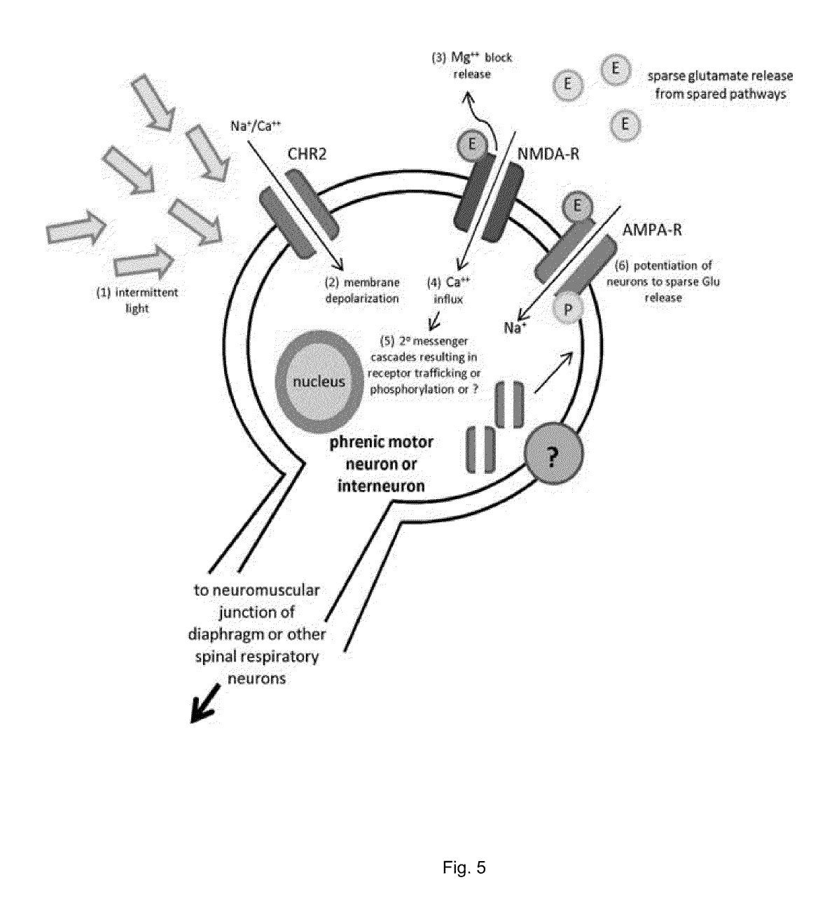

One component that plays an important role in activity dependent synaptic plasticity, learning, and adaptation in the CNS is the glutamatergic NMDA receptor. Our observation that the NMDA receptor antagonist MK-801 eliminated cycling of increasing diaphragmatic EMG activity after photostimulation begins to suggest a mechanism underlying this form or respiratory plasticity, recovery, and synaptic strengthening (FIG. 5). Because the NMDA receptor is a voltage-gated ionotropic glutamate receptor, the depolarization caused by photostimulation of ChR2 could result in release of the Mg²⁺ ion blocking the channel (FIG. 5). Once released of this block, Ca²⁺ influx can occur and a series of signaling cascades can begin leading to activation of the protein kinase C/RAF/MAP kinase sequence, and/or the SRC/Grb2/Sos sequence. In turn, both of these pathways can lead to initiation of ERK, increased protein synthesis, and/or immediate early gene translation. Ca²⁺ can also enter directly via ChR2 during light stimulation adding to these processes. Regardless of the downstream molecular cascade that might be involved, the NMDA receptor has been identified as a primary mediator of learning and long-term potentiation (LTP) in the hippocampus, in the induction of another mechanism of respiratory plasticity known as long-term facilitation (LTF), and in the spontaneous respiratory recovery observed after C2 hemisection. During the initiation of LTP and LTF, these forms of plasticity require intermittent stimulation and the plasticity we have uncovered may be analogous to or use the same cellular machinery as these events.

From these experiments, we can begin to hypothesize that there is a subthreshold level of patterned glutamate being released from spared pathways, because the NMDA receptor also requires glutamate binding to be activated along with membrane depolarization (FIG. 5). This sparse glutamatergic transmission may be potentiated on either phrenic motor neurons, interneurons or both through increased receptor presence on the postsynaptic membrane, phosphorylation of present receptors, or some totally new mechanism (FIG. 5). Other voltage-gated Ca²⁺ channels, such as the L/N/P/Q/T types, may also play a role in our observations and account for the limited response we saw in two MK-801-treated animals (FIG. 4B). Finally, the fine tuning of EMG activity may be mediated through activated Ca²⁺ SK channels which accompany NMDA receptor activation and LTP.

We have demonstrated that activation of C3-C6 spinal neurons, including denervated phrenic motor neurons or interneurons, some with contralateral projections, through stimulation of the ChR2 protein can restore repeatedly, diaphragmatic muscle activity that is rhythmic and persistent even after the cessation of light. This is the first time that this emerging technology has been successfully used after traumatic CNS injury to restore activity. Our data suggests that after debilitating lesions of the CNS, which leave CNS neurons denervated and quiescent, incorporation of the algal protein ChR2 (as well as the hyperpolarizing off-switches, vertebrate rhodopsin 4 or halorhodopsin) and subsequent photostimulation of infected neurons is a possible alternative to restore and control neuronal activity, possibly through potentiation of denervated target neurons and supersensitivity to spared axonal inputs. In the case of SCI, which can leave entire spinal motor neuron pools with zero or only minimal amounts of supraspinal input, this exciting and potential therapy is one that should be further explored and studied. With the perfection of an in vivo light source, it can be envisioned that more chronic manipulation and stimulation of spinal neurons or neuronal circuits, including spinal central pattern generators through light, can lead to recovery of lost function after SCI including bowel and bladder function and possibly walking to improve the quality of life of SCI patients.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

Having described the invention, the following is claimed:

1. A method of improving functional efficacy of a denervated, quiescent, or dormant motor neuron, the method comprising:
expressing one or more light sensitive G protein coupled receptors in the motor neuron, the one or more light sensitive G protein coupled receptors modulating cellular activity in the motor neuron upon exposure to a wavelength of light; and
exposing the motor neuron expressing the one or more light sensitive G protein coupled receptors to the wavelength of light.

2. The method of claim 1, the modulation of cellular activity stimulating bursting activity in the motor neuron.

3. The method of claim 1, expressing one or more light sensitive G protein coupled receptors in the motor neuron comprising transfecting the motor neuron with a vector construct, the vector construct including a nucleotide encoding at least one light sensitive G protein coupled receptor and a promoter.

4. The method of claim 1, wherein the exposure to a wavelength of light comprises patterned intermittent photostimulation.

5. The method of claim 1, the motor neuron comprising a phrenic motor neuron.

6. The method of claim 1, the motor neuron comprising an Onuf's nucleus neuron.

7. The method of claim 1, the one or more light-sensitive G protein coupled receptors comprising at least one of channelrhodopsin, vertebrate rhodopsin, or invertebrate rhodopsin.

8. The method of claim 1, the one or more light-sensitive G protein coupled receptors selected from the group consisting of channel rhodopsin 2, vertebrate rhodopsin 4 and combinations thereof.

9. A method of restoring functional breathing in a subject with a Central Nervous System (CNS) injury comprising:
expressing one or more light sensitive G protein coupled receptors in motor neurons that affect functional breathing in the subject, the one or more light sensitive G protein coupled receptors modulating cellular activity in the motor neurons upon exposure to a wavelength of light; and
exposing the motor neurons expressing the one or more light sensitive G protein coupled receptors to the wavelength of light.

10. The method of claim 9, the potentiation of cellular activity stimulating bursting activity in the motor neurons that affect functional breathing.

11. The method of claim 9, expressing one or more light sensitive G protein coupled receptors in the motor neurons comprising transfecting the motor neurons with a vector construct, the vector construct including a nucleotide encoding at least one light sensitive G protein coupled receptor and a promoter.

12. The method of claim 9, wherein the exposure to a wavelength of light comprises patterned intermittent photostimulation.

13. The method of claim 9, the one or more light-sensitive G protein coupled receptors comprising at least one of channel rhodopsin, vertebrate rhodopsin, or invertebrate rhodopsin.

14. The method of claim 9, the one or more light-sensitive G protein coupled receptors selected from the group consisting of channel rhodopsin 2, vertebrate rhodopsin 4 and combinations thereof.

15. A method of improving bladder function in a subject, the method comprising:
expressing one or more light sensitive G protein coupled receptors in neurons that affect the bladder function, the one or more light sensitive G protein coupled receptors modulating cellular activity in the neurons upon exposure to a wavelength of light; and
exposing the neurons expressing the one or more light sensitive G protein coupled receptors to the wavelength of light.

16. The method of claim 15, the neurons that affect bladder function selected from the group consisting of neurons of an intradural nerve, an extradural nerve, a pudendal nerve, a pelvic nerve, a foraminal nerve, a dermatome and combinations thereof.

17. The method of claim 15, wherein modulating cellular activity in the neurons can include inhibiting cellular activity in the neurons.

18. The method of claim 15, wherein modulating cellular activity in the neurons can include promoting cellular activity in the neurons.

19. The method of claim 15, expressing one or more light sensitive G protein coupled receptors in the neurons comprising transfecting the neurons with one or more vector constructs, the one or more vector constructs including a nucleotide encoding a light sensitive G protein coupled receptor and a promoter.

20. The method of claim 15, wherein the exposure to a wavelength of light comprises patterned intermittent photostimulation.

21. The method of claim 15, wherein the exposure to a wavelength of light comprises concurrently applying a first series of intermittent light pulses to neurons affecting external urethral sphincter (EUS) contractions and a second series of intermittent light pulses to neurons affecting bladder contractions, wherein the first and second series of intermittent light pulses are synchronized to mitigate interference with one another and to reduce or eliminate EUS contractions and evoke bladder contractions to expel urine from the subject.

22. The method of claim 21, wherein the first and second series of intermittent light pulses have a substantially same on time for corresponding light pulses of the first and second series of intermittent light pulses.

29. The method of claim 21, wherein the first and second series of intermittent light pulses have a substantially same on time and off time period for corresponding light pulses of the first and second series of intermittent light pulses.

23. The method of claim 15, the one or more light-sensitive G protein coupled receptors comprising at least one of a channel rhodopsin, a vertebrate rhodopsin, or an invertebrate rhodopsin.

24. The method of claim 15, the one or more light-sensitive G protein coupled receptors comprising channel rhodopsin 2.

25. The method of claim 15, the one or more light-sensitive G protein coupled receptors selected from the group consisting of vertebrate rhodopsin 4, halorhodopsin, and combinations thereof.
